(19) United States
(12) Patent Application Publication
(10) Pub. No.: US 2006/0120914 A1
(43) Pub. Date: Jun. 8, 2006

(54) Machine and method for treating products with microwaves

(76) Inventor: Luciano Salda, Vignola (IT)

Correspondence Address: STITES & HARBISON PLLC, 1199 NORTH FAIRFAX STREET, SUITE 900, ALEXANDRIA, VA, 22314, US

(21) Appl. No.: 10/559,686

(22) PCT Filed: Jun. 16, 2004
(86) PCT No.: PCT/EP04/06472

§ 371 Date: Dec. 6, 2005

(30) Foreign Application Priority Data
Jun. 30, 2003 (IT) BO2003A000402

(51) Int. Cl. A61L 2/00; H05B 6/64

(52) U.S. Cl. 422/21; 219/678

(57) ABSTRACT

The machine comprises a plurality of identical containers (1), of suitable shape and suitable capacity, preferably having flared mouths and round cross sections, which, by the action of a carousel (7) or any other suitable conveyor means, are cyclically made to interact with the following operating stations: a first station which shreds the containers with their contents, in such a way as to form a finely shredded product which, by suitable means, is fed into each container in predetermined constant amounts, is compacted and is suitably moistened, the mouth of each container being protected and kept clean by suitable means; a second station in which the mouth of the container (1) is closed to form a seal by a piston (39) which communicates by means of a wave guide with the magnetron (55) which generates the necessary microwaves for the sterilization of the waste in the container. Suitable means control the pressure and temperature within the container, in such a way that the waste is treated at the specified temperature and for the time required to ensure neutralization of any bacterial load present in the waste, while on completion of sterilization, the treatment container is brought to atmospheric pressure and removed from the said piston; a station, if required, for removing any excess liquid contained in the sterilized waste, while the mouth of the container is suitably protected and kept clean; and a station which discharges the treated waste from the container, while the mouth of the said treatment container is protected and kept clean by suitable means, so that it can subsequently interact and form a seal with the piston of the sterilization station.

The systems used at present for the microwave treatment of products in general, and of waste in particular, are usually of the continuously operating type and require continuous supply of the said waste to a treatment chamber where the said waste can be kept temporarily or can be moved by the action of conveyor means, and where they are heated by the action of the microwaves to the desired temperature and for the desired time, and are then discharged continuously to means of disposal. These system are expensive and difficult to operate, and are unsuitable for cyclical treatment of the variable quantities of solid waste produced, for example, in a hospital environment, which at present are collected in suitable cardboard or corrugated plastic boxes and which are intended for disposal in authorized incinerators. There are also intermittently operating microwave treatment systems, typically used in laboratories, which are not organized on the basis of automatic supply and discharge of waste to and from the treatment chamber.

In relation to the specific problem of sterilizing hospital waste or similar problems, in order to ensure that the said waste can be neutralized in a limited time after its formation, with advantages in the containment of the propagation of any bacterial load of the waste, and to ensure that after treatment the said waste can be disposed of at low cost in the same way as ordinary waste, the invention proposes an easily controlled cyclically operating machine using highly reliable technology, based substantially on the following proposed solution. The machine comprises a plurality of identical containers, of suitable shape and capacity, preferably with flared mouths and round cross sections, which, by the action of a carousel or any other suitable conveyor means, are cyclically made to interact with the following operating stations: a) a first station which shreds the boxes with the waste, to create a finely shredded product which, by suitable means, is fed into each container in predetermined, constant amounts, is compacted, and is suitably moistened, the mouth of the container being protected and kept clean by suitable means; b) a second station in which the mouth of the container is closed to form a seal by a piston which communicates by means of a wave guide with the magnetron which generates the microwaves required for the sterilization of the waste in the container. Suitable means control the pressure and if necessary the temperature within the container, in such a way that the waste is treated at the specified temperature and for the time required to neutralize any bacterial load present in the waste, while on completion of sterilization the treatment container is brought to atmospheric pressure and removed from the said piston; c) a station, if required, for removing any excess liquid contained in the sterilized waste, while the mouth of the container is suitably protected and kept clean; and d) a station which discharges the treated waste from the container, while the mouth of the said treatment container is protected and kept clean by suitable means, so that it can subsequently interact and form a seal with the piston of the sterilization station.

Further characteristics of the present invention, and the advantages derived therefrom, will be made clearer by the following description of a preferred embodiment of the invention, illustrated purely by way of example and without restrictive intent in the figures on the attached sheets of drawing, in which:

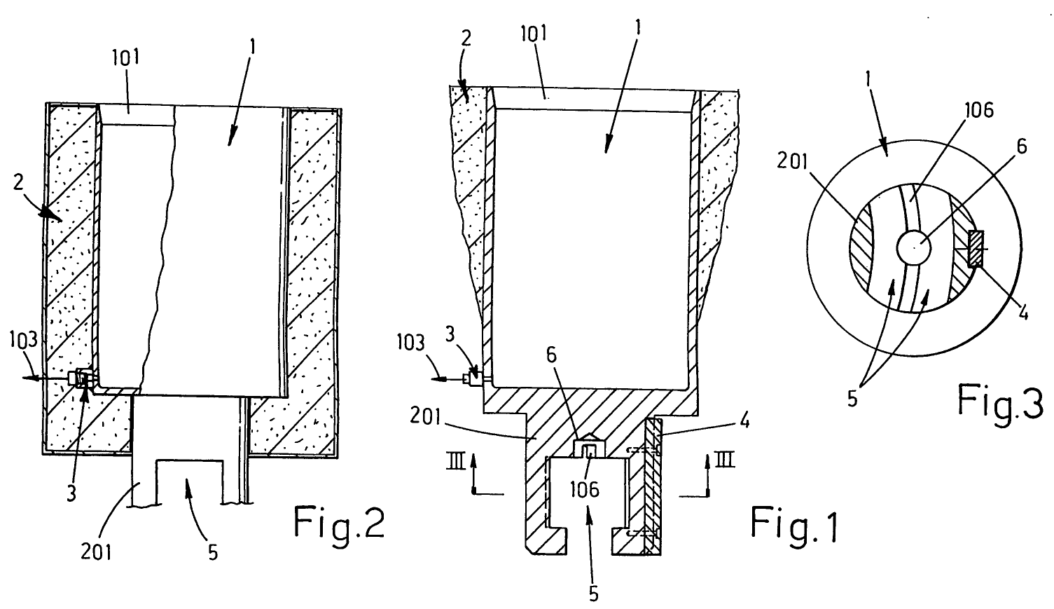

FIGS. 1 and 2 show in longitudinal section, with parts in view and with various details of construction, one of the treatment containers used by the machine;

FIG. 3 shows other details of the container of FIG. 1, in a section taken through the line III-III;

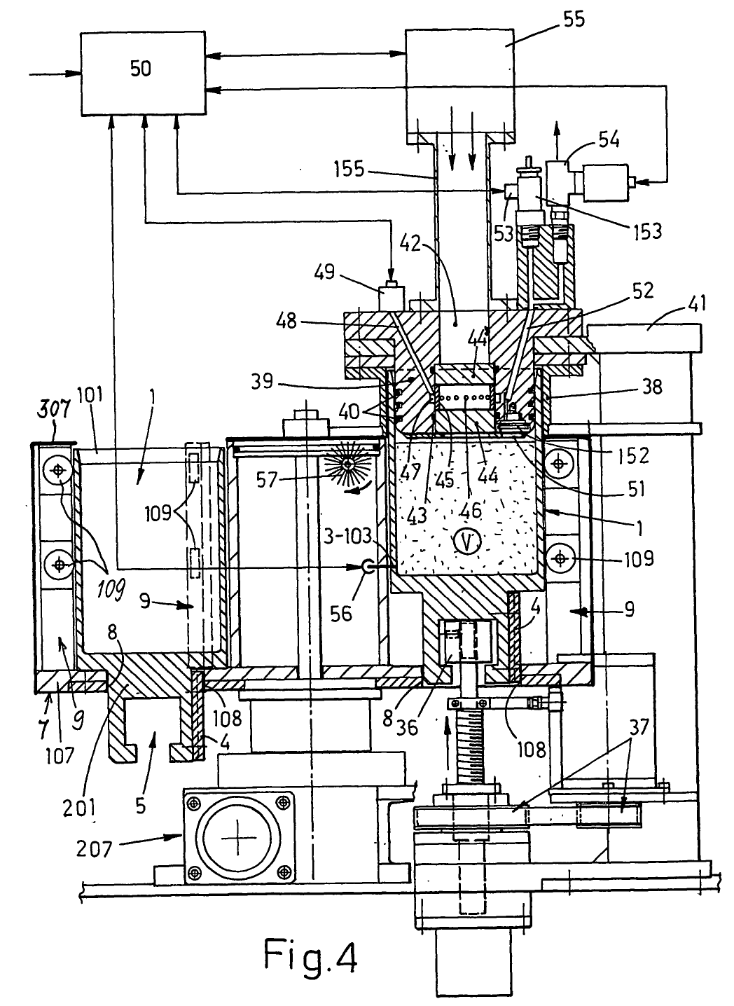

FIG. 4 shows in a side view and with parts in section the carousel on which the containers shown in the preceding figures are mounted, and shows, in particular the waste sterilization station;

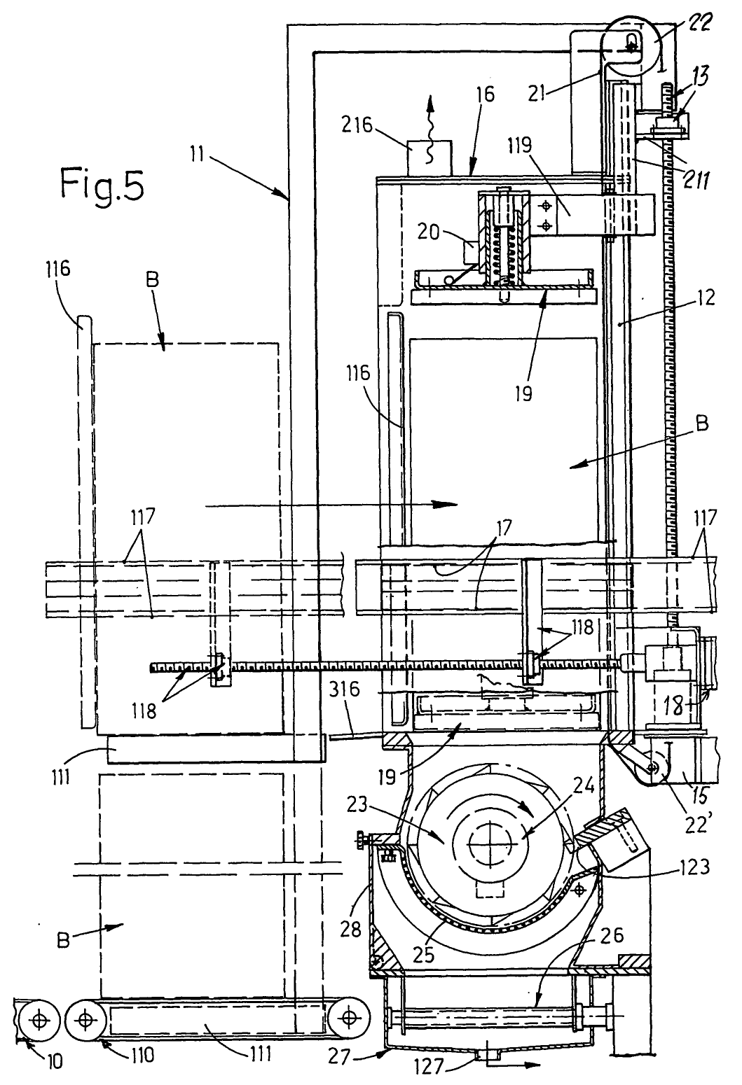

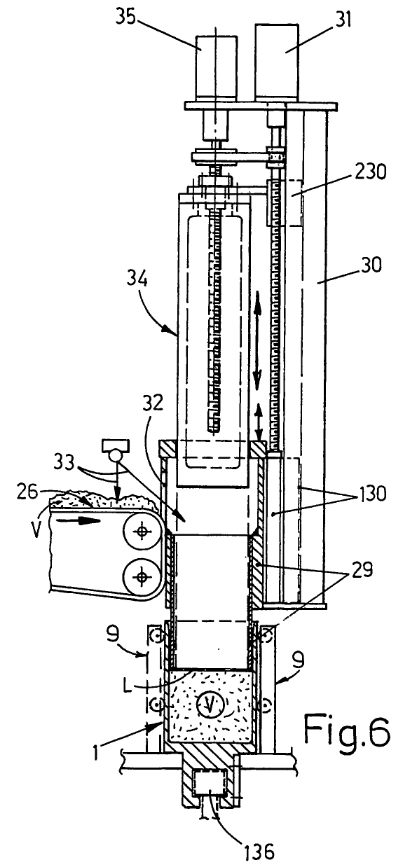

FIGS. 5 and 6 show, in front and side elevation respectively, with parts in section, the initial and final parts of the station for shredding the waste and for feeding them into the treatment containers with appropriate moistening;

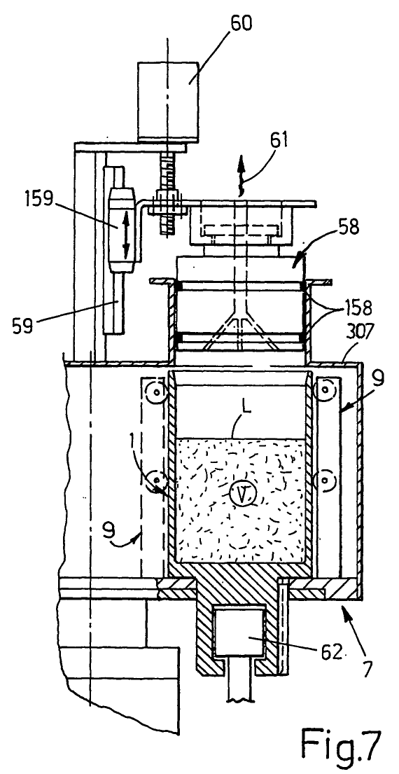

FIG. 7 shows, in a side elevation with parts in section, the station for drying the treated waste;

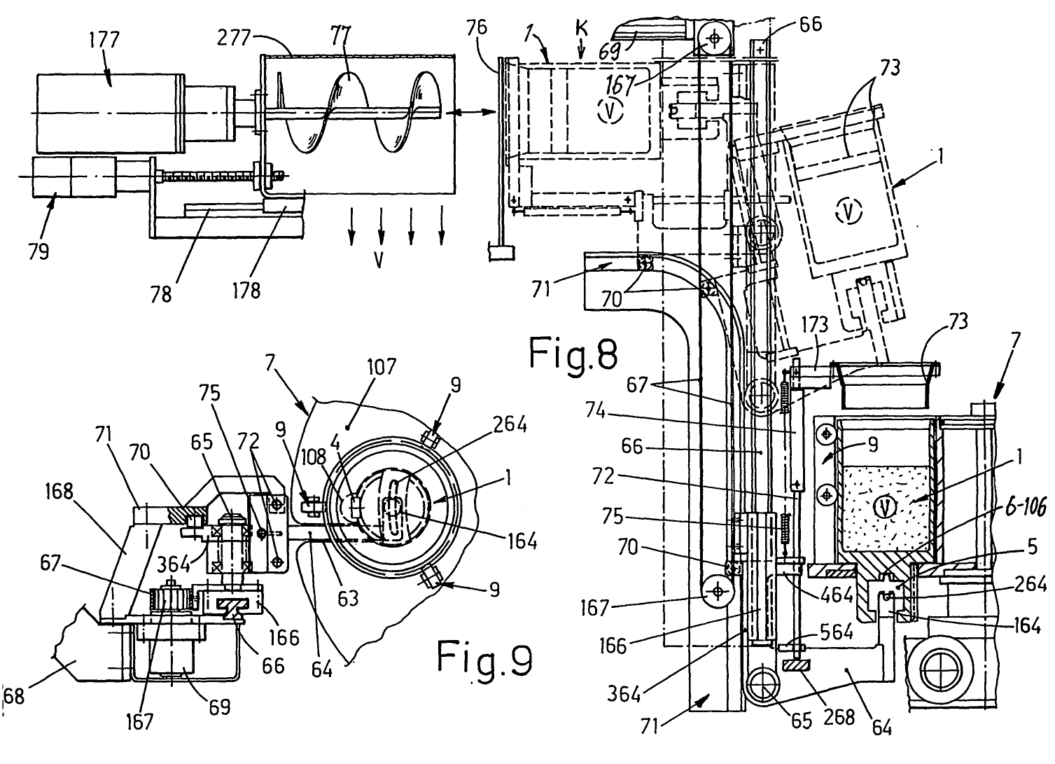

FIGS. 8 and 9 show, in side elevation and in plan view from above respectively, the station for discharging the waste from the treatment containers and for cleaning the said containers, if necessary, before their return to the operating cycle.

FIG. 1 shows how the machine uses at least four or three (see below) cylindrical steel containers 1, with suitable dimensions, for example with an internal diameter of approximately 130 mm and with a height of approximately 190 mm, with suitably rounded lower internal angles and with an internally flared upper edge 101, preferably having an outer jacket 2 for thermal insulation (see also FIG. 2) and having if necessary, at least at the bottom, a housing for a temperature probe 3 which, for example, has a suitable plug 103 projecting from the said jacket 2 (see below). To allow for the type of handling for which they are intended in the present example, each of the containers 1 has an external axial shank 201 on its base with a lateral key 4 and with an underlying median aperture 5 in the form of a C rotated through ninety degrees, with a curve profile in plan view (FIG. 3) and having in the centre of its arched structure a round recessed indentation 6, intersected diametrically by a linear longitudinal median recessed indentation 106. For simplicity of representation, the containers 1 are illustrated without their jackets 2 in the other figures of the drawing. The containers 1 are mounted, for example, vertically and at equal angular intervals, on a carousel 7 with a vertical axis, as shown in FIG. 4, which is rotated on command, by means of its intermittent rotation system 207, with an angular amplitude equal to that present between the containers 1 located on it. The carousel 7 comprises a platform 107 with holes 8 suitably shaped for the passage of the shanks 201 and with enlargements 108 (see also FIG. 9) for the passage of the keys 4 of the containers 1, which thus rest with their bases on the said platform 17 and have their apertures 5 in vertical radial planes with respect to the carousel 7 and therefore always orientated correctly with respect to the stationary means which are required to engage with them cyclically and then disengage from them (see below). For this purpose, the curvature in plan view of the aperture 5 has its centre on the axis of the carousel 5. Vertical bars 9, spaced at equal angular intervals, with free-running rollers 109 having horizontal axes, are fixed on the said platform 107, at least three of these bars being provided for each container 1, to form true guide cages for the said containers. The carousel 7 can also be provided with a suitable casing 307 and other means to allow the various components of the carousel to be cleaned conveniently if necessary before each maintenance operation performed on it, for example by the introduction into the said casing of water vapour and/or other products suitable for the purpose, the whole arrangement being evident to persons skilled in the art and suitable for implementation by them in various ways. With reference to FIGS. 5 and 6, it will be noted that the first station with which the containers 1 are made to interact is that designed for loading the waste to be treated into the said containers. This station can be associated with means which automatically feed the boxes of waste to the shredding device. The boxes B filled with waste are, for example, placed in Indian file on a conveyor 10 which feeds the leading box to a final accelerating conveyor 110 which separates this box from the following one and which positions it correctly on top of the pair of parallel horizontal prongs 111 of an elevator 11. It should be understood that, in reality, the conveyor system 10, 110 can be rotated through ninety degrees from the position shown in FIG. 5, still with the final conveyor 110 positioned between the prongs of the elevator 11, but perpendicularly to these. The elevator 11 is associated with a slider 211 which slides on a vertical guide 12 and which is moved, for example, by a male and female thread system 13 driven by a reversible motor 15, assisted by limit microswitches which are not shown. The aforesaid guide 12 is fixed laterally to a parallelepipedal hopper 16 which is rectangular in plan, is large enough to contain at least one box B, and has a lateral hatch 116, whose height is greater than that of a box B, associated with a horizontal assembly consisting of a guide and slider 17, 117, the said slider being connected for example to a male and female thread drive system 118 powered by a reversible electric motor 18 and by means of limit microswitches which are not shown. An arm 119 fixed to the slider 211 enters the hopper 16 through a longitudinal aperture formed in the lateral wall opposite that closed by the hatch 116, and supports in the hopper a sprung horizontal presser 19, with a sensor 20 which senses the load of the springs and which is described more fully below. The ends of a band 21 of steel or other material, which closes the aperture of the hopper through which the said arm passes, and which runs around free-running stationary pulleys 22, 22′, are fixed to the arm 119. At the base of the hopper 16 there is provided at least one shredder 123 with fixed blades which interact with the peripheral blades of a shredding cylinder 23 which rotates about its own horizontal axis, usually in the clockwise direction with respect to FIG. 5, and which is driven by a reversible electric motor 24 with thermal protection. Under the rotor 23 of the shredder there is located a static or vibrating screening grid 25, which allows only the shredded waste V with the predetermined particle size to pass through, together with any liquid present in the waste. A conveyor means 26 of any type suitable for the purpose, which if necessary can allow any excess liquid to flow into an underlying tank 27 from whose discharge pipe 127 the said liquid is drained and can, for example, be circulated to moisten new material is placed in a parallel manner underneath the grid 25. The conveyor 26 can consist of a belt, perforated or porous if necessary, or a system with one or more vibrating channels in a cascade formation. The base of the hopper 16 is connected by means of a casing 28 to the tank 27, to prevent any dispersion of dust due to the shredding operation. For this purpose, it is possible to provide, in the upper part of the hopper 16 and/or in any other suitable position, ducts 216 connected to suction, filtration and sterilization means, which maintain a precise negative pressure in the whole shredding system. The station described above operates as follows. When the presser 19 is at the lower limit of its travel and at the point of maximum closeness to the shredder 23, the prongs 111 are positioned below and at the sides of the conveyor 110, which is then automatically activated to position a box B containing waste for disposal on top of it and above the said prongs 111. Weighing and monitoring means for other purposes can be provided in the are of the conveyor 110, to check whether or not the content of the box conforms to the safety conditions specified for correct operation of the machine, and, if these conditions are not present, means (not shown) are provided to remove the box from the conveyor 110. The top of the box placed on the conveyor 110 is located at a height such that it does not interfere with the lower part of the hatch 116 which is located above it and which, in the next step, is translated horizontally into the position shown in broken lines, to open the hopper 16 in which the shredder 23 has previously been stopped. In the next step, the slider 119 is raised to lift both the presser 19 and the elevator 11, and the upward travel is stopped when the presser is in the upper part shown in solid lines and the prongs 111 are in a position of substantial alignment with the base of the hopper 16. At this point, the motor 18 is restarted to translate the hatch 116 towards the hopper 16, and in this step the hatch itself acts as a pusher and transfers the box B into the said hopper, making it slide on the prongs 111 and then on a bridge 316 which is, for example, integral with the said hopper. When the box B has been fed in, the shredder 23 starts and the motor 15 is reversed to lower the presser 19 which executes its pushing action on the box B, to keep it progressively in correct interaction with the said shredder which gradually shreds it together with all of its contents. The descent of the presser 19 is stopped automatically when the sensor 20 detects that the springs with which the said presser is provided are stressed, and restarts automatically when the said sensor detects the opposite condition and the travel continues to the lower limit which is detected by a limit microswitch. If the thermal device controlling the motor 24 detects excessive force during the step of shredding the box B and its contents, means are provided to ensure that this motor stops, that the presser 19 is suitably raised and that the said motor then restarts in reverse, in such a way as to free the shredder. After a predetermined time, the shredder starts to rotate again in the clockwise direction and the presser 19 starts to descend again to restart the normal operating cycle. The operation of the shredder is clearly also controlled by means (not shown) which monitor the state of accumulation of the shredded waste on the conveyor 26, for example when this conveyor is stopped if necessary after the step of filling a treatment container 1. FIG. 6 shows that when the carousel 7 positions a container 1 in the filling station, the said container is placed under and in axial alignment with a sleeve 29 mounted on a slider 130 which slides on a vertical fixed guide 30 and which is connected to raising and lowering means consisting, for example, of a male and female thread unit, a reversible motor 31 and limit microswitches (not shown). On command, the sleeve 29 is lowered in such a way that its lower end engages in the mouth of the container 1 to protect it from contamination and to act as a funnel for connection to the means of feeding the waste to be sterilized. This is because the sleeve 29 is provided with a lateral aperture 32, aligned with the conveyor 26, which, when the said sleeve is lowered, is placed under the active branch of the said conveyor, while, when the sleeve is raised, the said aperture is raised and the sleeve also acts as a means of stopping the shredded waste placed on the conveyor 26. When the sleeve 29 is in the low position, the finely shredded waste V is discharged from the conveyor 26 into the container 1. Suitable means, indicated schematically by the arrow 33, are provided to spray a precise quantity of liquid, for example water, on to the waste V which is fed into the container 1 in the filling step. The upper end of the sleeve 29 is normally engaged by the lower end of a cylindrical piston 34, whose diameter is slightly less than the internal diameter of the container 1, and whose upper end is connected to a slider 230 which slides, for example, on the guide 30 and which is connected to means of raising and lowering, of the male and female thread type for example and with a reversible electric motor 35 with electronic speed, phase and torque control, for example a brushless motor. In a cyclical way, the conveyor 26 is stopped and the piston 34 is lowered to compress the waste in the container 1, torque control being provided at least in the final stage, in such a way as to form a thoroughly compacted layer of the said waste in the treatment container, for example at a constant level indicated by L, by means of any suitable operating logic which can be easily implemented and determined experimentally. It is important that the waste V introduced into the container 1 should be sufficiently moist, and it is therefore necessary to take into account the capacity and activation time of the feed means 33; it is possible to act on the waste while it is still located on the conveyor 26, on the assumption that excess liquid will run out of the said waste by gravity, or the liquid level within the container 1 can be monitored and/or adjusted, for example by means located in the piston 34. For this purpose, it should be noted that in the final stage of compression of the waste in the container 1, the waste acts like a sponge which is squeezed by the piston 34, as a result of which the liquid contained in the said waste tends to rise to the surface. By using sensors (not shown) fitted in the piston 34, it will be possible to detect the quantity of liquid present in the pressed waste and by using special means in the supply system 33 it will be possible to regulate this quantity of liquid, the whole arrangement being evident to persons skilled in the art and easily applicable by them. The waste introduced into the container 1 can be moistened in a different way from that illustrated, by using means located in the piston 34 which supply the liquid to the waste during the active strokes of the said piston which compress the said waste in the treatment container, instead of by using the aforesaid supply systems 33. When the container 1 has been filled, and after the piston 34 has returned to its upper rest position, the sleeve 29 is also raised, removed from the container 1 and returned to the start of cycle position. The weight of the container 1 when filled with waste is such that the container is kept in the low position by gravity, even if there is a slight degree of friction between the sleeve 29 and the mouth of the said container. If necessary, however, suitable means shown in broken lines and indicated by the number 136 can be provided on the base of the carousel to engage the lower aperture 5 of the shank of the container 1, to prevent the container 1 from following the sleeve 29 in the raising step and also to oppose the thrust exerted by the piston 34 in the step of compressing the waste, in order to avoid the anomalous discharge of this thrust on to the rotating platform 107 of the carousel 7.

With reference to FIG. 4, it will be seen that, while the carousel 7 positions a new container 1 in the aforesaid filling station, the container 1, which is leaving this station and is filled with the compacted mass of waste V, is transferred to the sterilization station which comprises, in particular, a mushroom-headed device 36 whose head engages in the lower aperture 5 of the shank of the container 1 and which, under the action of axial means of movement, for example means of the male and female thread type with a reversible motor 37, is raised through a predetermined necessary distance to push the upper part of the said container 1 into a protective and shielding sleeve 38 which surrounds, with a degree of clearance, a cylindrical piston 39 which enters, with a seal formed by its lateral gaskets 40, into the mouth of the said container 1 until it is close to the level L of the mass of waste V to be treated, this assembly 38, 39 being integral with a fixed support structure 41 which is fastened, for example, to the base of the carousel. The piston 39 has an axial cavity 42 of suitable shape and size, which terminates below in an enlarged portion in which are housed, with the interposition of corresponding lateral sealing gaskets 43, a pair of diaphragms 44, 44′ made of quartz or other suitable material which is permeable by microwaves but not by fluids, kept at the requisite distance from each by an annular spacer 45 having radial through holes 46 which communicate with an outer annular recess 47 to which is connected an ascending channel 48 formed in the body of the piston and connected to a pressure transducer 49 which transmits the electrical signal corresponding to its reading to a processor 50 which controls the operation of the machine or of the unit in question. The quartz pieces 44, 44′ are, for example, secured in place by a flange 51 fixed on the lower flange of the piston 39, but it should be understood that other suitable means can be used for this purpose. A pressure outlet 152 opens on the lower face of the piston 39 and is connected to an ascending channel 52 which branches into at least two passages, one of which is connected to a maximum pressure valve 153 and in parallel to a pressure transducer 53 which transmits the corresponding electrical signal to the processor 50. The other passage of the channel 52 leads to a solenoid valve 54 which is controlled by the said processor 50 and which discharges into a protected area, for example into the container (not shown) from which water is drawn for moistening the waste V during the filling of the containers 1. The axial cavity 42 of the piston 39 is connected by means of a waveguide 155 to the generator 55 which produces the microwaves required for the sterilization step, for example a conventional cavity magnetron of suitable power which operates in the frequency band around 2.45 GHz. To optimize the operation of the generator 55, the generator can if necessary be provided with known means which read the amount of waves reflected towards the said generator. The operation of the generator 55 is also controlled by the processor 50 which, through a circuit connected to a socket 56 which can be provided for connection to the plug 103 of the container 1, if present, can also receive the electrical signal relating to the temperature at the base of the said container 1 inserted into the sterilization station. The presence of the temperature probe 3, 103 is optional. It was provided in the prototype of the machine in question, in order to monitor in a safe way the operation of the said machine and to optimize the sterilization cycle, since the graph of sterilization as a function of time is not linear. In the production of the machine on an industrial scale, the said temperature probe can be omitted, since this would yield evident advantages in the construction and handling of the containers 1, and since the temperatures within the said containers can be deduced from the parameter of pressure which is directly proportional to the temperature and correlated with it. As the temperature rises, the liquid contained in the waste evaporates and the pressure increases. The station shown in FIG. 4 operates in the following way. When the container 1 has been coupled to the piston 39, the processor 50 causes the source 55 to be activated. For the sterilization step, a predetermined temperature must be reached within the container 1 and this temperature must be maintained for a predetermined time. For example, in a moisture-saturated environment, a temperature of approximately 150° C. maintained for approximately 9-10 seconds causes the assured destruction of any bacterial load present in the mass of waste in the container 1, without any combustion phenomena occurring in this mass. In the absence of the temperature control 56, 103, 3, when the processor 50 detects, through the pressure sensor 53, the attainment of a specified pressure level which significantly corresponds to the attainment of the said temperature, for example a pressure of approximately 8 bars, the source 55 is kept active for the aforesaid period of maintenance, which allows the temperature to be spread uniformly throughout the container, plus the sterilization time for the specific temperature, while the pressure within the container 1 is maintained around the said specified level by the modulated opening of the solenoid valve 54 which discharges water vapour. However, in the presence of the temperature control 56, 103, 3, which is located in the most critical part of the container 1, when the processor 50 detects the attainment of the specified temperature in the container, the source 55 is kept active for the time required for sterilization in the most critical part monitored by the temperature control system, while the pressure within the container 1 is maintained around a specified level, approximately 8 bars for example, by the modulated opening of the solenoid valve 54 which discharges water vapour. At the end of the sterilization cycle, the source 55 is switched off, the solenoid valve 54 is opened to discharge the residual pressure, and finally the container 1 is lowered and removed from the piston 39. During the active stage of operation of the sterilization station, if the pressure in the container 1 exceeds predetermined safety levels, the maximum pressure valve 153 is triggered. On the other hand, if the sensor 49 detects a pressure level above zero, the sterilization unit is depressurized and opened, and an alarm signal is generated to indicate the occurrence of the problem which is probably caused by the breaking of the seal 43 of the lower quartz piece 44 or by the breaking of the quartz piece itself. When the container 1 has been lowered, the carousel 7 rotates through ninety degrees, and a brush, or other suitable means 57 mounted on the said carousel, passes under the lower quartz piece 44 of the sterilization piston 39 to clean it. It should be understood that, in order to limit the reflection of the microwaves towards the generator 55, the latter can be positioned in any suitable way which may differ from what is illustrated, and that the bases of the containers 1 and/or the lower surface of the piston 39 can be shaped in a form which is not flat and which is suitable for the purpose, for example in a curved form, the whole arrangement being evident to persons skilled in the art and easily applied by them.

The operating station following the sterilization station can comprise means for removing the water from the sterilized waste V, this water being possibly recovered for the steps of moistening future waste to be treated. These means are shown in FIG. 7, and comprise a piston 58 with sealing gaskets 158, mounted on a slider 159 which slides on a vertical fixed guide 59 and which can be raised and lowered by means of a male and female thread system and a reversible electric motor 60, this motor being of the type with electronic speed and phase control if required, in such a way that it can be operated with appropriate acceleration and deceleration gradients. The piston 58 is gas-permeable and is connected to a vacuum pump represented schematically by the arrow 61 and to valve means (not shown), by means of which, when the piston 58 is inserted into the mouth of the container 1, the liquid contained in the waste can be drained off and the said container can then be returned to atmospheric pressure before the step of extraction and removal of the said piston 58 to the rest position. The aperture 5 of the shank 201 of the container can advantageously be engaged by the head of a mushroom-headed device 62 mounted on the base of the carousel, to keep the said container axially stationary in opposition to the axial movement which may be caused by the steps of insertion and extraction of the piston 58 and by the cavitation phenomena created by the vacuum pump 61. To improve the dehydration action provided by the vacuum pump and to ensure efficient cooling of the treated waste, each treatment container can be provided in its base with at least one one-way valve (not shown), which opens automatically in this step only, the whole arrangement being evident to persons skilled in the art and easily applicable by them. The station of FIG. 7 is followed by the station for discharging the sterilized waste V from the container 1, as will now be described with reference to FIGS. 8 and 9. FIG. 9 shows that the platform 107 of the carousel 7 has slots 63 opening on its periphery and having their other ends opening tangentially into the seats 8 for housing the shanks 201 of the containers 1. When a container 1 reaches this station, the aperture 5 of its shank 201 is entered by a vertical pin 164 which carries a curved cross-piece 264 running across its end, these components being aligned with the recesses 6 and 106 respectively on the top of the said aperture 5 (FIG. 3). The said pin 164 is integral with a small arm 64 aligned vertically with the said slot 63 and pivoted transversely at 65 on a carriage 166 which slides on a vertical fixed guide 66 and which is associated with a raising and lowering means, for example the vertical branch of a toothed belt conveyor 67 running around the pulleys 167 supported rotatably by the structure 68 which also supports the said guide 66, one of these pulleys being connected to and driven by a reversible electric motor 69, preferably of the type with electronic speed and phase control. When viewed from the side as in FIG. 8, the arm 64 is L-shaped, with one portion 364 orientated upwards and having fixed to its upper end a cross-piece 464 which is T-shaped in plan view, whose foot carries a rotatable roller 70 with a horizontal axis, which runs in a rectilinear fixed guide 71 having an appendage 168 fixed to the frame 68. The guide 70 has a large lower vertical rectilinear portion, followed by a portion curving away from the carousel and a short final portion which is rectilinear and practically horizontal. The end of the cross-piece 464 carries guide seats on its enlarged part above the arm 64, and corresponding guide seats are provided in a gusset 564 fixed to the said arm, and a pair of vertical bars 72 slide in these seats, the upper ends of these bars being fixed to the appendage 173 of a small hopper 73 which is held at a small distance from the container 1 to be emptied, for example by the interaction of the lower end of at least one of the said bars 72 with a fixed appendage 268, or by another suitable method. The hopper 73 is provided with a cylindrical lower part, designed for insertion with a good seal into the mouth of the container 1 to be emptied, and its upper end terminates in a conical and outward-diverging part. Tubes 74 of adequate height, acting as spacers in the way described below, are mounted on the portions of the bars 72 lying between the appendage 173 and the cross-piece 464. The number 75 indicates at least one cylindrical helical spring having one end fixed to the said appendage 173 and the other end fixed to the cross-piece 464, in such a way as to keep the hopper 73 pushed towards the arm 64. When a container 1 reaches the discharge station, the processor of the machine causes the motor 69 to be started in order to raise the carriage 166. The arm 64 rises and, with the pin 164 and the key 264, engages with the corresponding recessed parts of the base of the container 1 and raises the container which slides on its external guides 9. The hopper 73 remains stationary and is inserted into the upper mouth of the container 1 as the latter is raised. When the cross-piece 464 touches the lower end of the tubes 74, the hopper 73 has completed its travel for insertion into the container 1 and follows the latter in the raising step. Being held securely by the hopper 73 and by the lower unit 164, 264, the container 1 leaves the corresponding guides 9 of the carousel, and in the next step the roller 70 starts to interact with the curved part of the guide 71, as a result of which any further raising of the slider 166 causes a rotation of the container 1 about the pivot 65, this rotation ending when the said container reaches the horizontal position indicated by K and in broken lines, and when the hopper 73 touches the aperture of a vertical fixed protector 76, associated with means of collecting and evacuating the sterilized waste, these means not being shown, since they are irrelevant to the understanding of the invention. When the container 1 has reached the said horizontal position, it is aligned axially with a screw 77 driven by a motor 177, provided with a fixed and downwardly open casing 277, this assembly being mounted on a slider 178, which slides on a horizontal guide 78 aligned with the axis of the said screw, and which is connected to advancing and withdrawing means consisting, for example, of a male and female thread assembly and by a motor 79, preferably of the type with electronic speed and phase control. At the correct time, the motor 177 and then the motor 79 are activated to rotate the screw 77 and to insert it into the container 1, in order to extract the sterilized waste from the container. The operating cycle of the screw can be that which is most suitable for the purpose. On completion of discharging, it is possible to provide a step of cleaning the container 1 with a rotary brush, with suction means and/or with any other suitable means which are not illustrated. On completion of the discharge of the waste from the container 1, the motor 69 is made to rotate in a direction which is the reserve of the preceding direction, to return the said container to the corresponding guides 9 of the carousel 7 and to make the gripping parts 73, 164, 264 also return to the rest position shown in FIG. 8 in solid lines, in such a way that the carousel 7 can rotate through ninety degrees to repeat the cycle. It is to be understood that the construction of the machine as described can be subjected to numerous variations and modifications, relating, for example, to the use of means other than a carousel for transferring the treatment containers to the various operating stations, for example means of the transfer device type. The use of these means could, for example, be useful for simplifying the machine, since the containers would be placed naturally in a horizontal position and even with an inverted orientation while running around the sprockets of the transfer device and during the travel along the lower branch of the said transfer device, where it would be possible to provide, together with means for emptying the containers 1, means for cleaning the said containers and for testing the operation of any temperature probes 3 fitted in them. Other variants can be provided in respect of the shredding station, the station for feeding the waste into the treatment containers, and the station for sterilizing the waste.

1) Machine for treating products with microwaves, particularly for sterilizing solid hospital waste which is usually stowed in suitable cardboard or corrugated plastic boxes, or other products having similar requirements, characterized in that it comprises a plurality of robust containers (1) of any suitable shape and capacity, open at one end at least and suitably pressure- and heat-resistant, which, by the action of a carousel (7) with a vertical axis or any other suitable conveyor means, are cyclically made to interact with at least the following operating stations: a first station which shreds the boxes with the waste, to create a finely shredded product (V) which, by suitable means, is fed and compacted in predetermined constant amounts, suitably moistened, into each container (1); a second station where the mouth of the container, with the compacted, constant and moistened mass of waste inside, is temporarily sealed by suitable means (39) which communicate by means of a wave guide (155) with a source (55) which generates the microwaves with the necessary power and for the necessary amount of time to sterilize the said moist mass of waste (V), this station being equipped at least with means for monitoring and controlling the pressure generated in the container during the microwave heating and with means for depressurizing the container at the end of the cycle; and a final station which discharges the sterilized waste from the container.

2) Machine according to claim 1, in which a third station for dehydrating or drying the sterilized waste to a sufficient degree is provided between the second and final stations.

3) Machine according to claim 1, in which means for cleaning the treatment containers (1) can be provided in the final operating station or after this station.

4) Machine according to claim 1, in which means can be provided for sanitizing the treatment container (1) and if necessary all parts of the machine which operate in contact with the waste to be sterilized, before the said parts are subjected to maintenance operations.

5) Machine according to claim 1, in which the treatment containers (1) can be provided with an outer jacket for thermal insulation (2).

6) Machine according to claim 1, in which each treatment container (1) carries at least one temperature probe (3) with a corresponding external outlet (103) for removable, rapid and temporary connection to the means which detect the temperature inside the said container during the step of sterilizing the waste contained in it, this probe being preferably positioned as closely as possible to the base of the said container.

7) Machine according to claim 1, in which each treatment container (1) consists of a steel cylinder of suitable dimensions, with a round cross section, having a suitable inner flare (101) on its upper mouth and a suitable rounding of the angular region of the connection between the side surface and the base of the said container.

8) Machine according to claim 1, in which the treatment containers (1) are mounted vertically and with their mouths upwards on a carousel with a vertical axis (7), with the possibility of sliding between vertical guides (9, 109) fixed on this carousel, and each of the said containers is provided externally on its base with an axial shank (201) which has a key (4) passing through the shaped through hole (8, 108) in the platform (107) of the carousel, below which the said shank (201) projects with a lower aperture (5) in the shape of a letter C rotated through ninety degrees, and which is kept by the presence of the said key on a theoretical plane containing the axis of rotation of the carousel, in such a way that this aperture can be engaged and disengaged correctly by the head of opposing and/or moving means in the shape of a mushroom or a T, located in the operating stations of the machine.

9) Machine according to claim 1, in which the first operating station comprises:
Means (10-18) for feeding the boxes with the waste (B), one at a time, into the loading hopper (16) of a shredder (23, 123), with a presser (19) which progressively and in a controlled way pushes the said box towards the shredding means;
Screening means (25) which allow only the waste shredded to a sufficiently fine and predetermined particle size to pass downwards;
Conveyor means (26) which collect the shredded waste leaving the said screening means and carry it to a position above the container (1) to be filled;
Means (27, 127) which collect and drain the excess liquid from the shredded waste;
Means (33) for correctly moistening the shredded waste to be inserted and/or already inserted into the treatment container (1);
A vertical sleeve (29) whose lower end is inserted temporarily by any suitable means for a precise distance into the mouth of the treatment container (1) which is stationary in the loading station, and which is provided with a lateral aperture (32) through which passes the shredded waste arriving from the said conveyor means (26);
A piston (34) mounted on a vertical slider (230) driven, for example, by a motor (35) with electronic speed, torque and phase control, which cyclically makes the said piston travel along the said guide and compress the shredded waste placed in the treatment container;
Means for cyclically loading a quantity of shredded waste into the treatment container (1), with values of height and density which are as far as possible constant and predetermined, and means for ensuring that this load of waste contains a sufficient quantity of water.

10) Machine according to claim 9, in which suitable means (216) can be provided to keep the whole environment in which the waste is shredded at a precise negative pressure with respect to the external environment, and to remove and recover in a safe and hygienic way any dust resulting from the said step of shredding the waste.

11) Machine according to claim 1, in which the station for sterilizing the load of shredded waste placed in the treatment containers (1) comprises:
Means (36, 37) for temporarily raising the container (1) with the waste to be treated in such way that a fixed piston (39) enters the said container with a lateral seal and for a precise distance, this piston having an axial cavity (42) which is connected by a wave guide (155) to the source (55) which produces the microwaves at the frequency and with the power required for the sterilization step;
In the lower part of the said axial cavity of the piston (39), at least two diaphragms (44, 44′) of microwave-permeable but not fluid-permeable material, such as quartz, are provided at a precise distance from each other, and the chamber formed between these two diaphragms, delimited by a suitable spacer (45), is designed for connection through suitable passages (46-48) to a pressure sensor (49) connected to the processor (50) which controls the operation of the system;
On the lower face of the said piston (39) there opens a pressure outlet (152) which is connected, by means of a channel (52) having at least two branches, to a safety valve or maximum pressure valve (153) and to a pressure sensor (53) connected to the said processor (50), and is connected to at least one discharge solenoid valve (54) which is also controlled by the said processor (50);
Any necessary means (56) for the temporary and removable connection of the processor (50) to any temperature probe (3, 103) which may be housed in the base of the treatment container (1);
Means (50) for activating the source of the microwaves (55) and for ensuring that, when the processor (50) detects the attainment of a predetermined temperature, by means of the temperature sensor (3) if present, or when it indirectly detects this value by means of the pressure sensor (53) which senses a corresponding value of the operating pressure, the said source (55) is kept active for a predetermined period which can be in accordance with the data acquired by the said temperature sensor and/or the pressure sensor, while the pressure within the container (1) is kept at around the said operating level by the modulated opening of the said solenoid valve (54), provision being made to ensure that, at the end of the cycle of sterilization of the waste placed in the treatment container, the said source of microwaves (55) is switched off, the said solenoid valve (54) is opened to discharge the residual pressure, and finally the said container (1) is removed from the piston (39) located above and is opened, provision being made to ensure that, during the active stage of operation of the station in question, if the pressure in the container (1) exceeds predetermined safety levels, the said safety valve (153) is triggered, and that, if the safety sensor (49) detects a pressure in excess of zero, the sterilization unit is automatically depressurized and opened, and an alarm signal is generated to warn of the occurrence of the problem, which is probably caused by the breaking of the seal (43) of the lower quartz piece (44) or by the breaking of this quartz piece.

12) Machine according to claim 2, in which the station, if any, operating after the sterilization station and responsible for drying or dehydrating the sterilized waste in the treatment container (1) comprises a piston (58) with corresponding external seals (158) and comprises means (62) for inserting this piston into the mouth of the said container and vacuum means (61) are connected to the said piston and to its channels for internal connection to the container, for sucking from the latter the excess liquid contained in the sterilized waste.

13) Machine according to claim 12, in which, in order to improve the operation of the said station for drying the treated waste, each treatment container (1) can be provided in its base with at least one one-way valve which opens automatically to allow the passage of ambient air only when the said container is connected to the said vacuum source (61).

14) Machine according to claim 1, in which the final station for discharging the sterilized waste from the treatment container (1) comprises:
Means (63-69) for raising the container (1) with the waste from the corresponding lateral guides (9, 109) with the coupling of anti-rotation means (164, 264) with corresponding recessed parts (6, 106) provided in the shank (201) of the said container, these raising means being associated with and opposed to an annular hopper (73) which by reaction to the raising of the container (1) and in opposition to the action of elastic means (75) is inserted into the mouth of the container to protect it from contamination and to form an externally enlarged and diverging extension of the said mouth, the said raising means being provided with means (70, 71) for making the container (1) rotate into a substantially horizontal position during the final part of the said raising;
Means, of a screw type for example (76-79), for extracting the treated waste from the container (1) in a substantially horizontal position, in such a way that the waste falls on to means of collection and evacuation;
Means for returning the emptied container (1), between the corresponding vertical guides (9, 109) of the carousel, until it rests on the platform (107) of the said carousel, with the original angular orientation, while the assembly with the hopper (73) interacts with fixed means (268) which stop its downward travel in such a way that the said hopper is disengaged from the container (1) and is suitably removed from it, to allow the free transfer of the container by the carousel (7) to the next operating station.

15) Machine according to claim 14, in which means can be provided in the final operating station or after this station, to clean the treatment containers internally and if necessary to test the operation of the temperature probes, if present (3, 103).

16) Machine according to claim 9, in which the sleeve (29) and the hopper (73), which are inserted into the mouth of the treatment container (1) during the loading and discharge of the waste into and from the said container, have such shapes and dimensions and are made from such materials that the whole of the upper inner part of the said container (1) designed to interact with the piston (39) of the waste sterilization station is kept clean.

17) Machine according to claim 8, characterized by the use of a transfer, with horizontal axes for example, in place of the carousel with the vertical axis (7), for cyclically transferring the treatment containers (1) to the successive operating stations, means being provided to retain the said containers in the corresponding guides (9, 109) during the stage of running around the sprockets of the transfer and during travel along the lower branch of the said transfer, means of discharging the shredded waste and means, if any, of cleaning the said containers being made to operate during the stage in which the containers are positioned horizontally and orientated downwards.

18) Process for treating products with microwaves, particularly for sterilizing solid hospital waste, usually stowed in suitable boxes (B), characterized by the succession of the following operating steps:
Fine shredding of the boxes with the waste and moistening with water, to precise levels, of the shredded product (V) obtained in this step;
Insertion of a precise quantity of shredded and moistened waste into treatment containers (1) open at one end at least, and compaction of this waste, in such a way that the load of waste has characteristics of height, density and moisture content which are as far as possible constant and predetermined;
Closing the said treatment container (1) to form a seal, and connecting the container to a source (55) which generates microwaves at approximately 2.45 GHz for example and with suitable power, to raise the temperature of the waste and water within the said container to precise levels and to maintain this temperature for a time sufficient to ensure the sterilization of the said waste, while the internal pressure of the container is regulated by a controlled discharge towards the exterior, in such a way that predetermined maximum values are not exceeded, the treatment container with the sterilized waste being made to be depressurized and reopened at the end of the cycle;
Discharge of the sterilized waste from the treatment containers, cleaning of the said containers (1) if necessary, and reintroduction of the containers into the operating cycle.

19) Process according to claim 18, in which the sterilization step includes keeping the shredded and moistened waste at a temperature of approximately 150° C. for a period of approximately 9-10 seconds at least, while the pressure within the treatment containers is kept below a specified maximum level, for example below 8 bars.
